(19) United States
(12) Patent Application Publication
(10) Pub. No.: US 2017/0056090 A1
(43) Pub. Date: Mar. 2, 2017

(54) APPARATUS AND METHOD FOR TREATING A BLOOD VESSEL

(71) Applicant: SIERRA MEDICAL INTERNATIONAL, INC., Irvine, CA (US)

(72) Inventors: JAY J. EUM, IRVINE, CA (US); HYUNG R. KIM, IRVINE, CA (US)

(73) Assignee: SIERRA MEDICAL INTERNATIONAL, INC., IRVINE, CA (US)

(21) Appl. No.: 15/242,333

(22) Filed: Aug. 19, 2016

Related U.S. Application Data
(60) Provisional application No. 62/209,283, filed on Aug. 24, 2015.

(51) Int. Cl. A61B 18/08

(52) U.S. Cl. CPC A61B 18/082; A61B 2018/00261

(57) ABSTRACT

The apparatus and method includes a proximal balloon subsystem including a proximal balloon positionable to a selected first location within a blood vessel to be ablated. A distal balloon subsystem including a distal balloon is positionable to a selected second location within the blood vessel to be ablated. An ablating subsystem including an ablating element is configured to be positioned between the proximal balloon and the distal balloon within the blood vessel to be ablated. An evacuation port is configured to evacuate fluid between the proximal balloon and the distal balloon.

### CROSS REFERENCE TO RELATED APPLICATIONS

This application claims the benefit of U.S. Provisional Application Ser. No. 62/209,283, filed on Aug. 24, 2015, the entire contents of which are hereby incorporated herein by reference thereto.

### BACKGROUND OF THE INVENTION

1. Field of the Invention

The present invention relates generally to treating blood vessels and more particularly to shrinking and/or clogging a blood vessel, such as the great saphenous vein.

2. Description of the Related Art

This invention is related to an apparatus and method for applying energy to shrink or clog blood vessels. The venous system of the lower extremities of human includes the superficial venous system and the deep venous system with veins connecting the two. The superficial venous system includes great saphenous and short saphenous veins.

The arteries carry the blood from the heart while the veins return the blood back to the heart. In the lower extremities, the direction of blood flow in the veins is against the gravitational force. Therefore, the veins of human body have one-way valves to prevent reverse flow of the blood, away from the heart. When these valves fail, the vein will not be able to close completely and the resulting leakage leads to varicose vein and chronic venous insufficiency conditions.

The condition of varicose veins is very obvious to the naked eye due to the swollen and twisted appearance of the veins just under the skin of the legs. Reflux within the great saphenous vein leads to pooling in the visible varicose veins below.

Most of time, there are no symptoms related to the varicose veins other than the visual appearance but when the symptoms worsen, patients can experience pain, blood clots, and skin ulcers.

Current treatment methods for the conditions include surgical stripping of the vein or closing the vein using heat energy or a clogging agent. By closing the vein, blood flow is redirected to other veins including deep veins. This eliminates the symptoms and visual swelling of the superficial veins. By closing the great saphenous vein, the twisted and varicosed branch veins, which are close to the skin, shrink and improve in appearance. Once the diseased vein is closed, other healthy veins take over to carry blood in the leg, re-establishing normal flow.

Surgical stripping of the veins was in practice longer than any other treatment methods but the catheter-based closure procedure is preferred due to the minimally invasive nature and faster recovery time.

Current heat-based closure procedures involve applying laser or RF energy to shrink the veins. The laser procedure has been in use longer than the RF procedure. However, a laser produces much greater heat than radio frequency and therefore requires a higher level of attention to perform. All laser procedures use forward firing laser fiber and require the user to pull the fiber at sufficient rate to insure proper treatment. Over treating at one spot or at an area can cause severe damage to the surrounding tissue.

One apparatus for an RF based heat procedure utilizes electrodes that expand out from the tip of the catheter. Once expanded, the electrodes touch the intima layer of the vein wall to create an impedance loop between the electrodes (bipolar) or between the electrodes and the ground pad (monopolar) to flow RF current. This causes the vein walls to heat up and shrink. With this apparatus the amount of current flow may significantly differ from case to case due to the differences in impedance, thus making this procedure difficult to control. This apparatus also requires the user to move the catheter along the vein to expand the treatment area. Furthermore, this apparatus requires moving the catheter while the electrodes are touching the vein wall with sufficient force to maintain the impedance level. This can cause sudden spikes of current or cold treatment zones if the impedance changes significantly while moving the electrodes.

Another apparatus for an RF procedure uses an enclosed impedance source at the tip of the catheter to produce heat. With this apparatus, since the catheter diameter is smaller than the inner diameter of the vein, the vein has to be squeezed externally to close the gap between the inner wall of the vein and the outer surface of the catheter. In order to achieve this, tumescent fluid is injected around the targeted area of the vein to compress the vein. Uniform compression of the vein is dependent upon the user and the injection technique employed. Although continuous pulling and moving of the catheter is not required like the aforementioned apparatus, the catheter still needs to be pulled back for the length of the treatment zone to expand the treatment area and ensure uniform treatment.

As will be disclosed below the present invention obviates requirements of the above.

### SUMMARY OF THE INVENTION

In one aspect, the ablating apparatus for use in treating a blood vessel of the present invention includes a proximal balloon subsystem including a proximal balloon positionable to a selected first location within a blood vessel to be ablated. A distal balloon subsystem includes a distal balloon positionable to a selected second location within the blood vessel to be ablated. An ablating subsystem including an ablating element is configured to be positioned between the proximal balloon and the distal balloon within the blood vessel to be ablated. An evacuation port is configured to evacuate fluid between the proximal balloon and the distal balloon.

The ablating element may use any number of modalities that are suitable for ablating a blood vessel. The ablating element may be a heating element. Examples include, but are not limited to, a resistive heater, a radio frequency electrode, a laser heating fiber, and a microwave antenna probe. If it is a resistive heater it may be, for example, a resistive wire heater, semi-conductor material heater or resistive sheath heater. The energy source may be, for example, direct current, alternative current, or radio frequency current. The ablating element may be a cooling element such as a cryosurgical device.

In another aspect, the present invention is a method for treating a blood vessel. A proximal balloon and a distal balloon of an ablating apparatus are positioned to selected spaced locations within a blood vessel to be ablated, the ablating apparatus including an ablating element positioned between the proximal balloon and the distal balloon within the blood vessel. The proximal balloon and the distal balloon are inflated to abut blood vessel walls of the blood vessel at the selected spaced locations. Fluid within the blood vessel between the proximal balloon and distal balloon is evacuated. The ablating element is powered to ablate the blood vessel to be treated, between the proximal balloon and the distal balloon.

The ablating apparatus can be used with an overall multi-functional control system for controlling the overall ablative process including, for example, monitoring the temperature of the ablating device, timing the ablation, and controlling the pressure in the evacuation port(s).

Other objects, advantages, and novel features will become apparent from the following detailed description of the invention when considered in conjunction with the accompanying drawings.

### BRIEF DESCRIPTION OF THE DRAWINGS

FIG. 1 is a perspective illustration of a first embodiment of the ablating apparatus for use in treating a blood vessel, of the present invention.

FIG. 2 is a longitudinal section taken along line 2-2 of FIG. 1.

FIG. 3 is an exploded sectional view showing the three subsystems separate.

FIG. 4 is an enlarged section of the proximal balloon.

FIG. 5 is an enlarged section of the proximal manifold.

FIG. 6 is an enlarged section of the distal balloon.

FIG. 7 is an enlarged section of the distal manifold.

FIG. 8 is an enlarged section of the heating manifold.

FIG. 9 is a section of two balloons in the blood vessel before blood is extracted.

FIG. 10 is a section of two balloons in the blood vessel after blood is extracted.

FIGS. 11A-11H are schematic views of the ablating apparatus of the first embodiment in operation.

FIG. 12 is a perspective illustration of a second embodiment of the ablating apparatus for use in treating a blood vessel, of the present invention, in which a long heating element is used.

FIG. 13 is a sectional view of the two balloons and the long heating element, taken along line 13-13 of FIG. 12.

FIGS. 14A-14I are schematic views of the apparatus of the second embodiment in operation.

FIG. 15 is a longitudinal sectional view of a third embodiment of the ablating apparatus for use in treating a blood vessel, of the present invention, in which the distal manifold and heating manifold are combined as a single piece.

FIG. 16 is enlarged section of the proximal balloon thereof.

FIG. 17 is an enlarged section of the proximal manifold thereof.

FIG. 18 is an enlarged section of the distal balloon thereof.

FIG. 19 is an enlarged section of the distal and heating manifolds thereof.

FIG. 20 is a longitudinal sectional view of a fourth embodiment of the ablating apparatus for use in treating a blood vessel, of the present invention, in which a second lumen is extruded into the injection tube system.

FIG. 21 is enlarged section of the proximal balloon thereof.

FIG. 22 is a view taken along line 22-22 of FIG. 21.

FIG. 23 is an enlarged section of the proximal manifold thereof.

FIG. 24 is an enlarged section of the distal balloon thereof.

FIG. 25 is an enlarged section of the distal and heating manifolds thereof.

FIG. 26 is a schematic illustration of an ablating system utilizing the ablating apparatus.

FIG. 27 is a perspective illustration of the fifth embodiment of the ablating apparatus for use in treating a blood vessel, of the present invention, in which a separate moveable evacuation subsystem is used.

FIG. 28 is a longitudinal sectional view of a fifth embodiment of the ablating apparatus for use in treating a blood vessel, of the present invention, in which a movable fluid extraction subsystem is incorporated into the treatment apparatus.

FIGS. 29A-29J are schematic views of the apparatus of the fifth embodiment in operation.

### DETAILED DESCRIPTION OF THE INVENTION

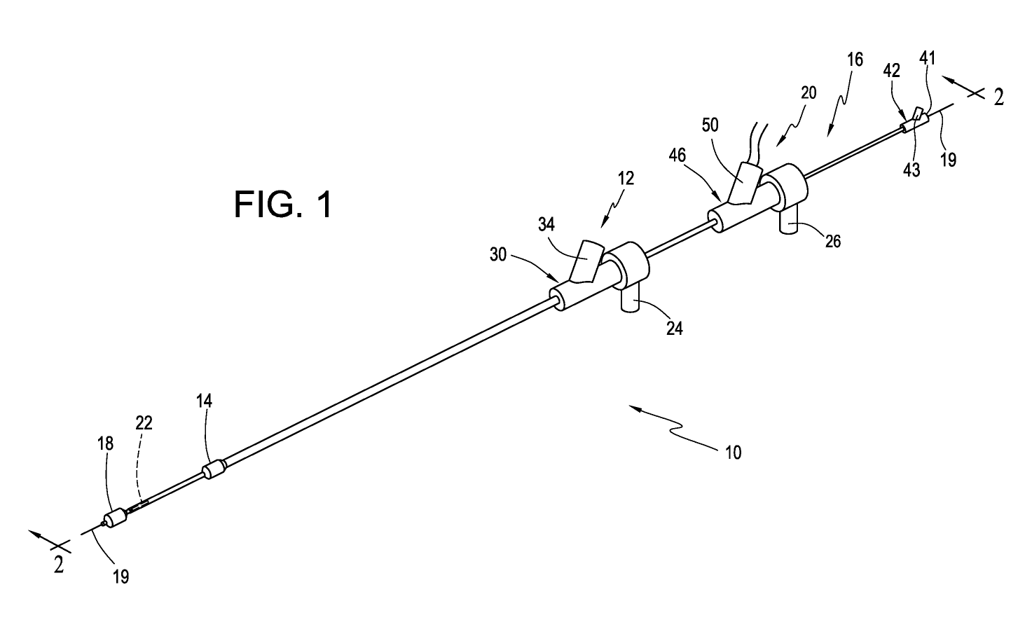

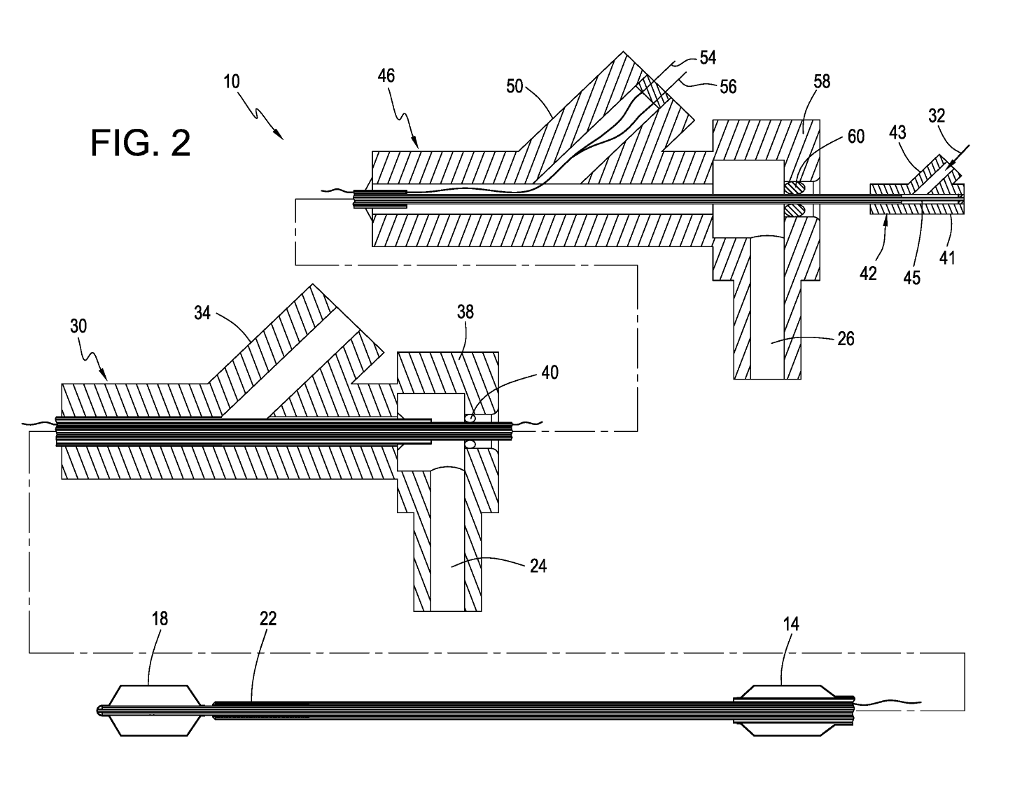

Referring now to the drawings and the characters of reference marked thereon, FIGS. 1 and 2 illustrate a first embodiment of the present invention, designated generally as 10. The apparatus 10 includes a proximal balloon subsystem 12 including a proximal balloon 14 positionable to a selected first location within a blood vessel to be ablated. A distal balloon subsystem 16 includes a distal balloon 18 positionable to a selected second location within the blood vessel to be ablated. An ablating subsystem 20 (e.g. heating subsystem) includes an ablating element 22 (e.g. heating element) configured to be positioned between the proximal balloon 14 and the distal balloon 18 within the blood vessel to be ablated. Evacuation ports 24 and 26 are configured to evacuate fluid between the proximal balloon 14 and the distal balloon 18. A guide wire 19 may be used to guide the apparatus 10 as will be discussed below.

The ablating element may use any number of modalities that are suitable for ablating a blood vessel. The ablating element may be a heating element. Examples include, but are not limited to, a resistive heater, a radio frequency electrode, a laser heating fiber, and a microwave antenna probe. If it is a resistive heater it may be, for example, a resistive wire heater, semi-conductor material heater or resistive sheath heater. The energy source may be, for example, direct current, alternative current, or radio frequency current. The ablating element may be a cooling element such as a cryosurgical device. Thus, in the various configurations illustrated herein, even though certain elements and subsystems may be referred to as “heating,” this is for the purposes of illustration and other forms of ablation can be utilized.

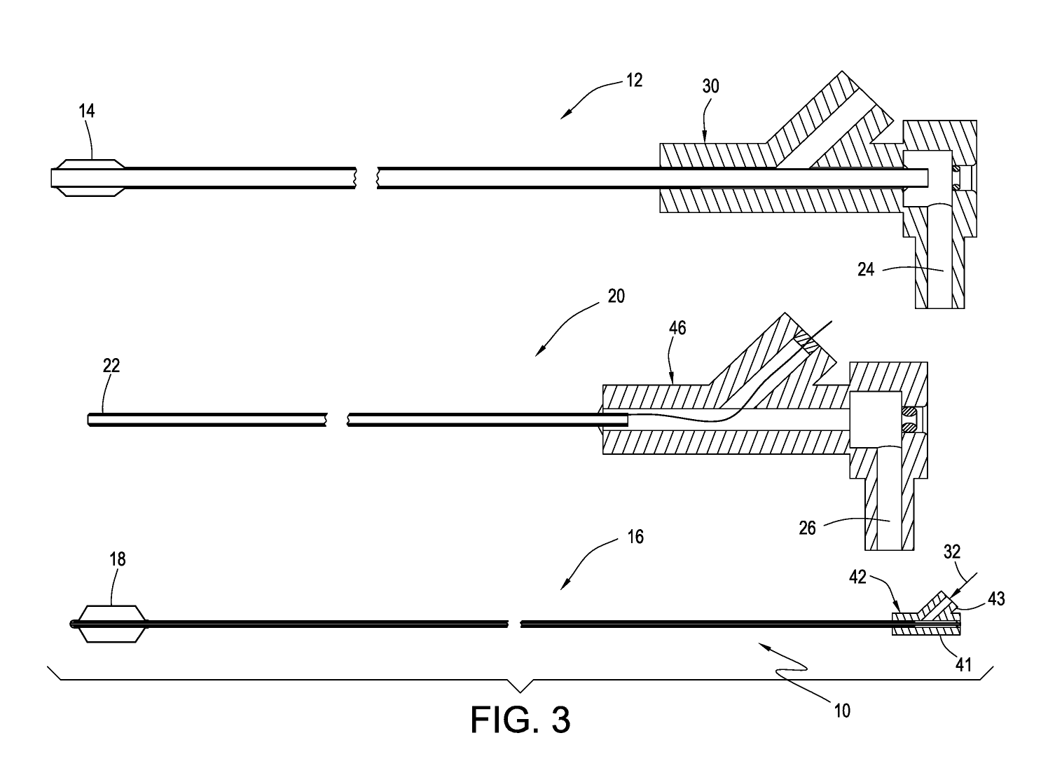

Referring also now to FIG. 3 the proximal balloon subsystem 12, distal balloon subsystem 16, and the ablating subsystem 20 (e.g. heating subsystem) are shown in an exploded view.

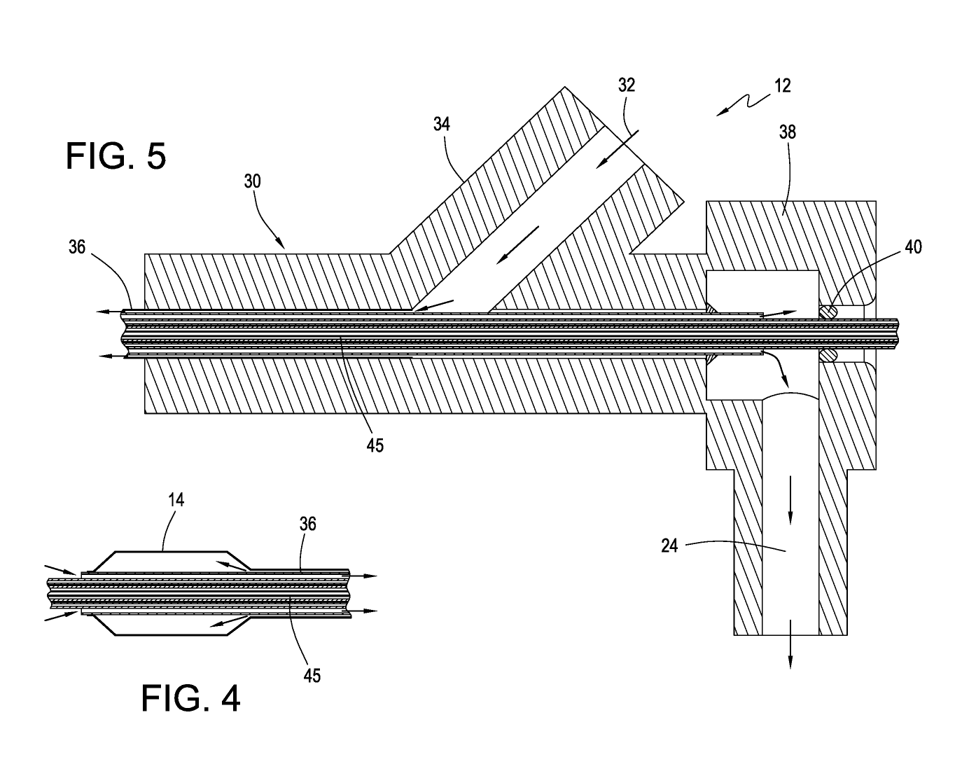

Also referring to FIGS. 4-5, the proximal balloon subsystem 12 includes a proximal manifold 30 configured to receive balloon inflating fluid 32. The proximal manifold 30 includes a proximal balloon injection port 34 for the balloon inflating fluid (generally a saline solution). A proximal balloon injection tube system 36 is attached to the proximal manifold 30 for transmitting the balloon inflating fluid 32 to the proximal balloon 14. The proximal balloon 14 is positionable to a selected first location within a blood vessel to be ablated.

A proximal balloon subsystem vacuum seal valve 38 with seal 40 provides evacuation of fluid within the blood vessel between the proximal balloon and the distal balloon.

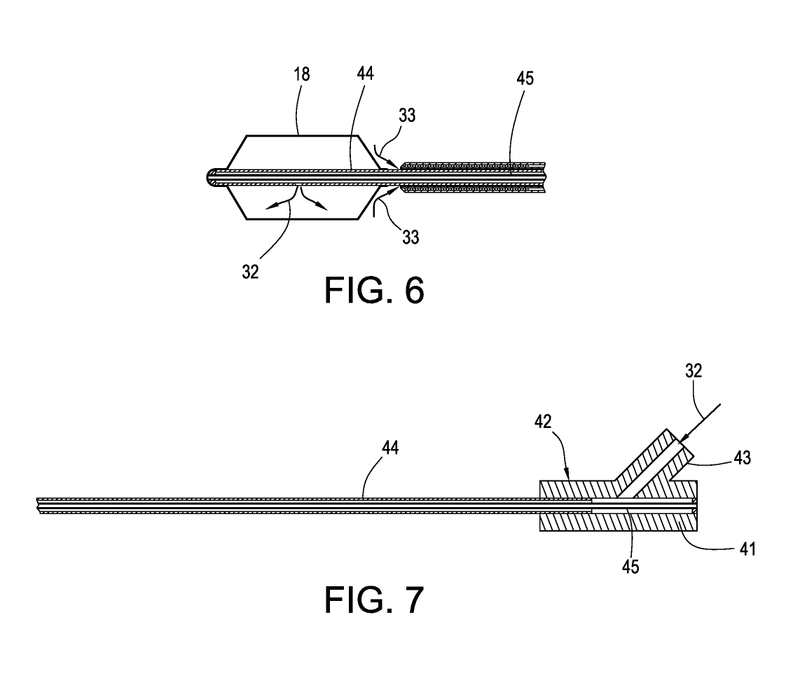

Referring now also to FIGS. 6 and 7 the distal balloon subsystem 16 includes a distal manifold 42 configured to receive balloon inflating fluid 32. The distal manifold includes a distal balloon injection port 43 and a guide wire port 41 for a guide wire tube 45. A distal balloon injection tube system 44 is attached to the distal manifold for transmitting balloon inflating fluid 32 and allowing the guide wire 19 to pass through the guide wire tube 45. The distal balloon injection tube system 44 is in fluid communication with the distal balloon 18. Arrows 33 indicate that fluid (principally blood) can be evacuated at the distal portion of the apparatus 10.

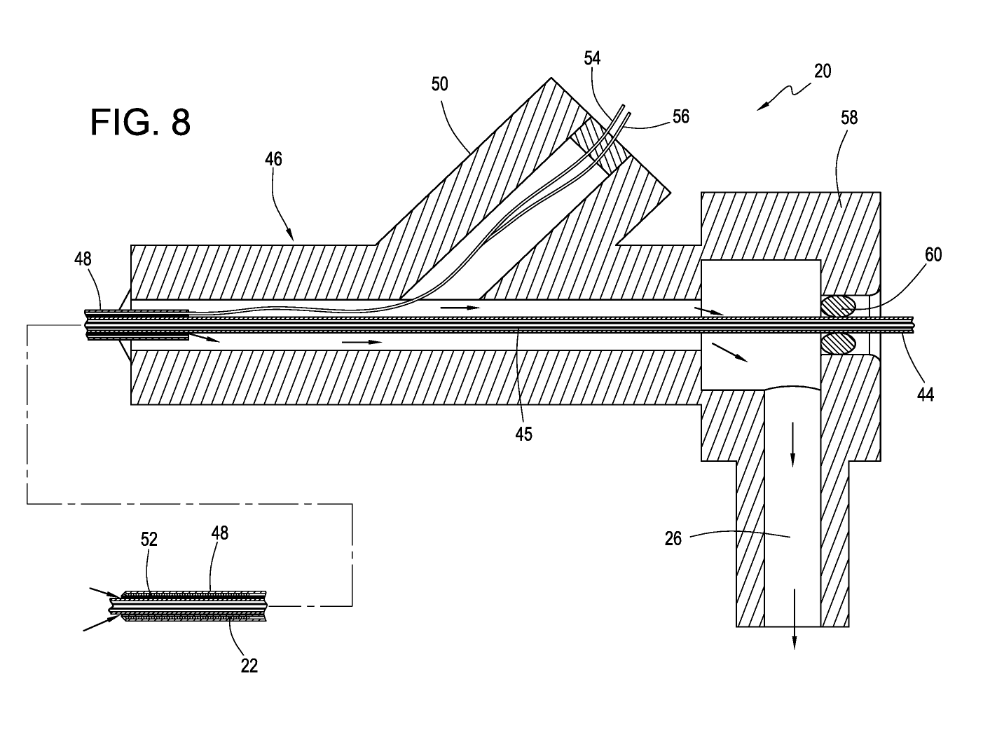

Referring now also to FIG. 8 the ablating subsystem 20 (e.g. heating subsystem) includes an ablating manifold 46 (e.g. heating manifold). An ablating element tube system 48 (e.g. heating element tube system) is connected to the ablating manifold 46. An ablating and sensing conduit 50 (e.g. heating and sensing conduit); and, ablating element tube system 48 (e.g. heating element tube system) provide access to a temperature monitoring element 52 (e.g. a thermocouple) through wires 54. The temperature monitoring element 52 is operatively connected to a distal end of the ablating element tube system 48. The ablating subsystem 20 includes the ablating element 22 operatively connected to a distal end of the ablating element tube system 48 to heat the blood vessel to be treated. Suitable wires 56 can be passed through the ablating and sensing conduit 50 for access to the ablating element 22.

An ablating subsystem vacuum seal valve 58 (e.g. heating subsystem vacuum seal valve) with seal 60 provides evacuation of fluid within the blood vessel between the proximal balloon 14 and the distal balloon 18. The evacuation of such fluid is indicated by the arrows in this figure.

The distal balloon injection tube system 44 is positioned within the ablating element tube system 48 and within the ablating manifold 46. The ablating element tube system 48 is positioned within the proximal balloon injection tube system 36. In this embodiment the proximal manifold 30 includes an evacuation port 24 and the ablating manifold 46 includes an evacuation port 26. However, it is understood that in other embodiments either one or both could have an evacuation port. The evacuation ports should be configured to provide sufficient suction to enable the blood vessel to shrink. The evacuation ports may be connected to an overall ablating system (as will be discussed below). Thus, vacuum pressure for blood and other fluids can be monitored to insure proper shrinkage of the vessel. The ablating system may also monitor temperature and provide energy for ablation. The ablating system is typically configured so that it will not energize the ablation element unless the proper vacuum level has been achieved and will discontinue energy flow if the vacuum level drops below a predetermined threshold. In other embodiments, a user can evacuate blood manually using a syringe while the pressure sensor or gauge, which is attached to the syringe, measures the vacuum level of the evacuation port.

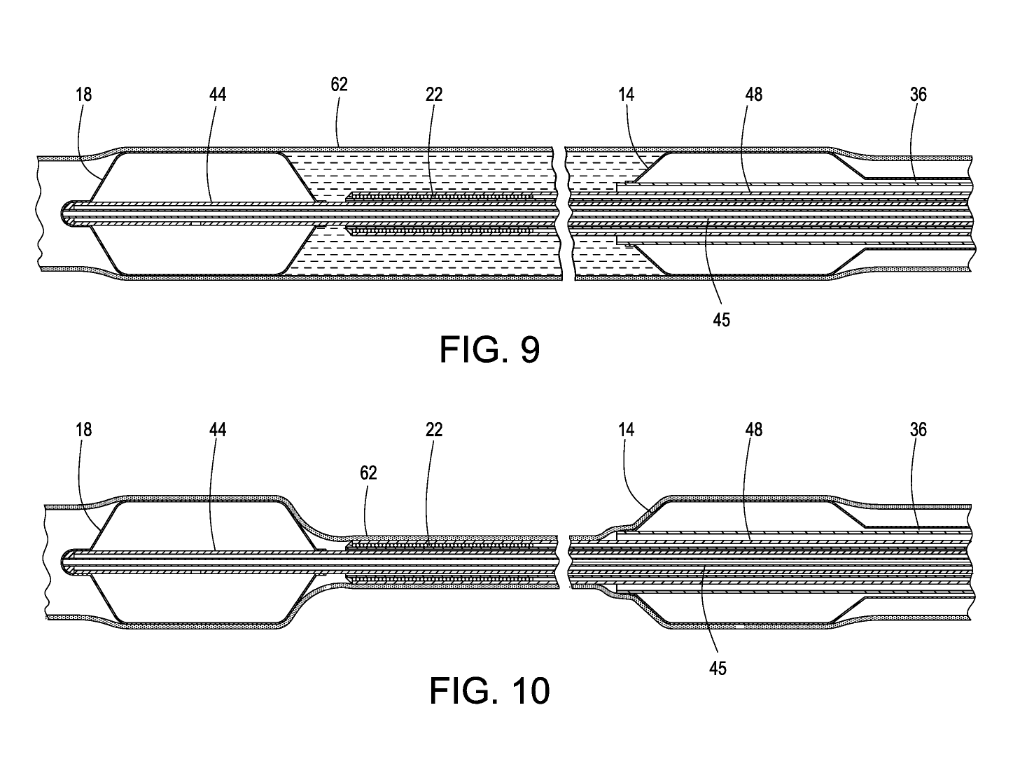

FIG. 9 shows two balloons in the blood vessel 62 before blood is extracted. FIG. 10 shows the balloons in the blood vessel after blood is extracted.

The ablating element 22 may be, for example, any number of suitable heaters. Examples include, but are not limited to, a resistive heater, a radio frequency electrode, a laser heating fiber, and a microwave antenna probe. If it is a resistive heater it may be, for example, a resistive wire heater, semi-conductor material heater or resistive sheath heater. The energy source may be, for example, direct current, alternative current, or radio frequency current. The ablating element may be a cooling element such as a cryosurgical device.

The proximal balloon 14 and/or the distal balloon 18, may be compliant, semi-compliant and non-compliant balloons. They may be formed of, for example, polyamide, Pebax® polyether block amide, polyethylene terephthalate (PET), polyimide, or polyurethane. The expanded diameter size of the proximal balloon 14 and the distal balloon 18 may be in a range of between about 2 mm-10 mm. The length of the distal balloon 18 and the proximal balloon 14 may be in a range between about 5 mm-30 mm. The thickness of the proximal balloon 14 and the distal balloon 18 may be in a range between about 0.005 mm to 0.080 mm.

The proximal balloon injection tube system 36, the distal balloon injection tube system 44, and the ablating element tube system 48 may be formed of biocompatible material, for example polyetheretherketone (PEEK), polythylene, TEFLON® polytetrafluoroethylene, polyamide, polyimide, Hytrel® thermoplastic elastomer, or Pebax® polyether block amide. The wall thickness of these tubes may be in a range of, for example, 0.001 in to 0.025 in.

The distal manifold 42, the proximal manifold 30, and the ablating manifold 46 may be formed of biocompatible material, for example, polyvinyl chloride (PVC), polyethylene, polyetheretherketone (PEEK), polycarbonate, polyetherimide (PEI), polysulfome, polypropylene, polyurethane, polyamide or polyimide.

The present invention is particularly useful for treating varicose veins; however, it can be used for treating a variety of blood vessels and conditions, including, for example, deep vein thrombosis (DVT), peripheral artery disease (PAD), and restenosis.

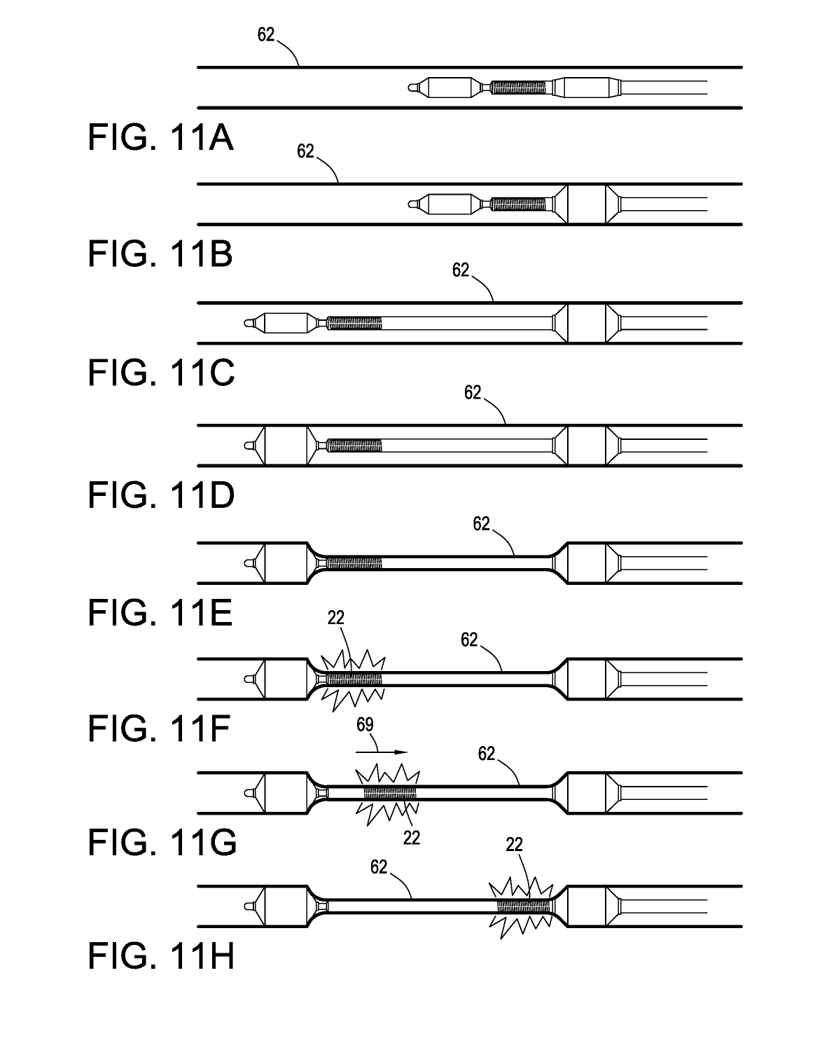

Referring to FIGS. 11A-D the method for treating a blood vessel includes an initial step of positioning a proximal balloon and a distal balloon of an ablating apparatus, to selected spaced locations within a blood vessel to be ablated. These setup steps include:

a) inserting the ablating apparatus to an initial position within the blood vessel (FIG. 11A);

b) inflating the proximal balloon to abut blood vessel walls of the blood vessel (FIG. 11B);

c) positioning the distal balloon to a distal location of the selected spaced locations (FIG. 11C); and,

d) inflating the distal balloon to abut blood vessel walls of the blood vessel (FIG. 11D).

As shown in FIG. 11E, fluid within the blood vessel between the proximal balloon and distal balloon is evacuated.

The ablating element is then powered. As shown in FIGS. 11F-11H the ablating element is slid along the apparatus between the distal balloon and the proximal balloon to ablate the blood vessel, as indicated by arrow 69.

Thus, the present invention eliminates the need for injecting tumescent fluid.

Furthermore, generally in prior systems for treating varicose veins there is a need to press down the skin using a hand. The present invention eliminates this need.

Injection of tumescent fluid typically results in irregular compression of the blood vessel. The present invention provides uniform blood vessel contraction.

Since there is enhanced contact between the blood vessel and the ablating element the ablation time is shortened relative to prior systems. Also, less energy is required.

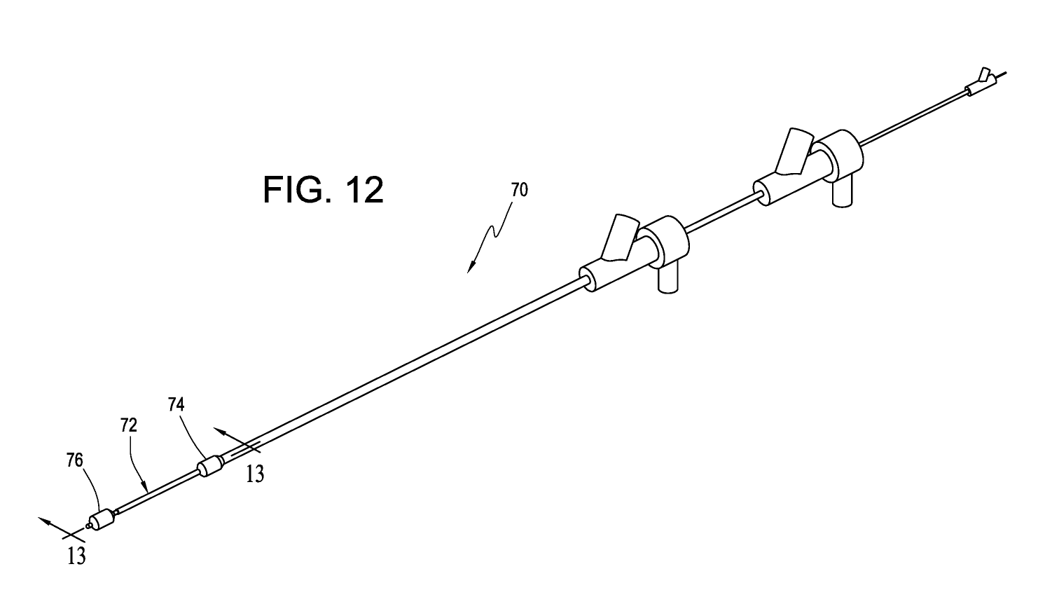

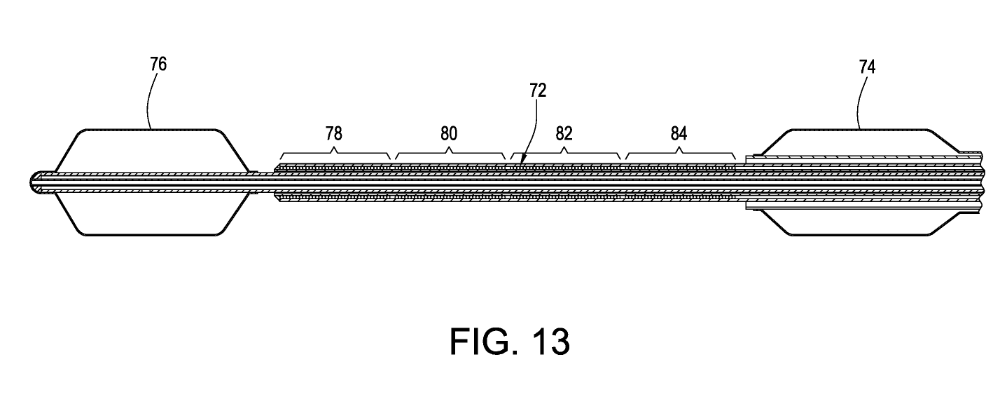

Referring now to FIGS. 12-13 a second embodiment is illustrated, designated generally as 70, in which a long heating element 72 is utilized which extends a substantial portion of the distance between the proximal balloon 74 and the distal balloon 76. Although this embodiment will be discussed relative to heating it is understood that other ablation modalities could be used, as discussed above, with this embodiment and the other embodiments discussed below. Segmented portions 78, 80, 82, 84 of the long heating element 72 between the distal balloon 76 and the proximal balloon 74 heat the blood vessel for selected periods of time. Segmented portions 78, 80, 82, 84 provide enhanced control and uniform heating during treatment.

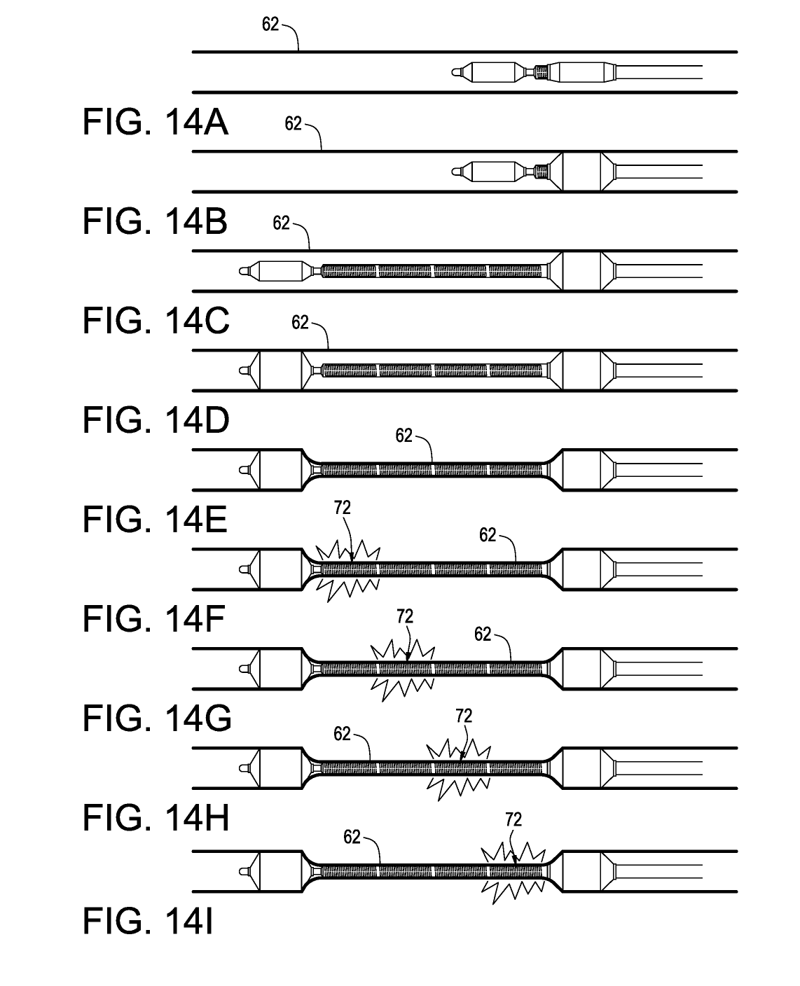

FIGS. 14A-14I illustrate the method of operating the second embodiment having a long, segmented heating element capable of activating each segment independently from each other for variable treatment length without moving the long heating element 72.

Referring to FIGS. 14A-D the method for treating a blood vessel includes an initial step of positioning a proximal balloon and a distal balloon of an ablating apparatus, to selected spaced locations within a blood vessel to be ablated. These setup steps include:

a) inserting the ablating apparatus to an initial position within the blood vessel (FIG. 14A);

b) inflating the proximal balloon to abut blood vessel walls of the blood vessel (FIG. 14B);

c) positioning the distal balloon to a distal location of the selected spaced locations (FIG. 14C); and,

d) inflating the distal balloon to abut blood vessel walls of the blood vessel (FIG. 14D).

As shown in FIG. 14E, fluid within the blood vessel between the proximal balloon and distal balloon is evacuated.

The ablating element is then powered. As shown in FIGS. 14F-14I the ablating element segments are powered sequentially to ablate the blood vessel.

Use of a long heating element with segmented portions obviates the need to move/reposition the apparatus 70 after every ablation. This enables a relatively quick ablation.

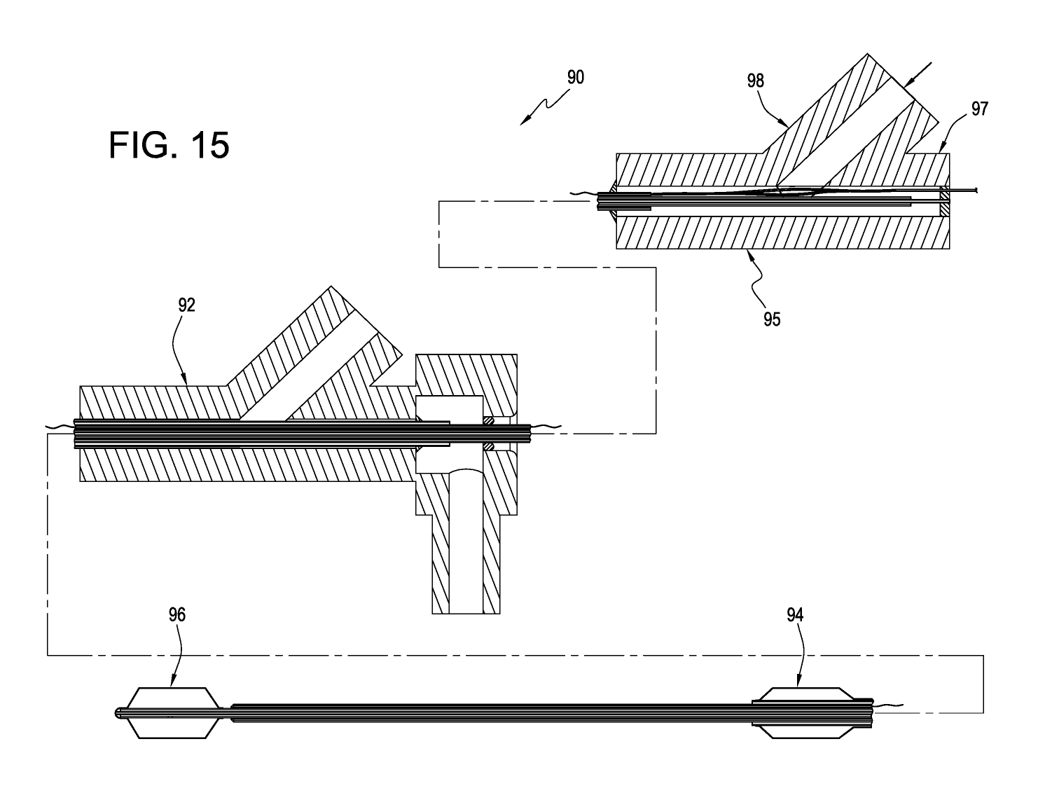

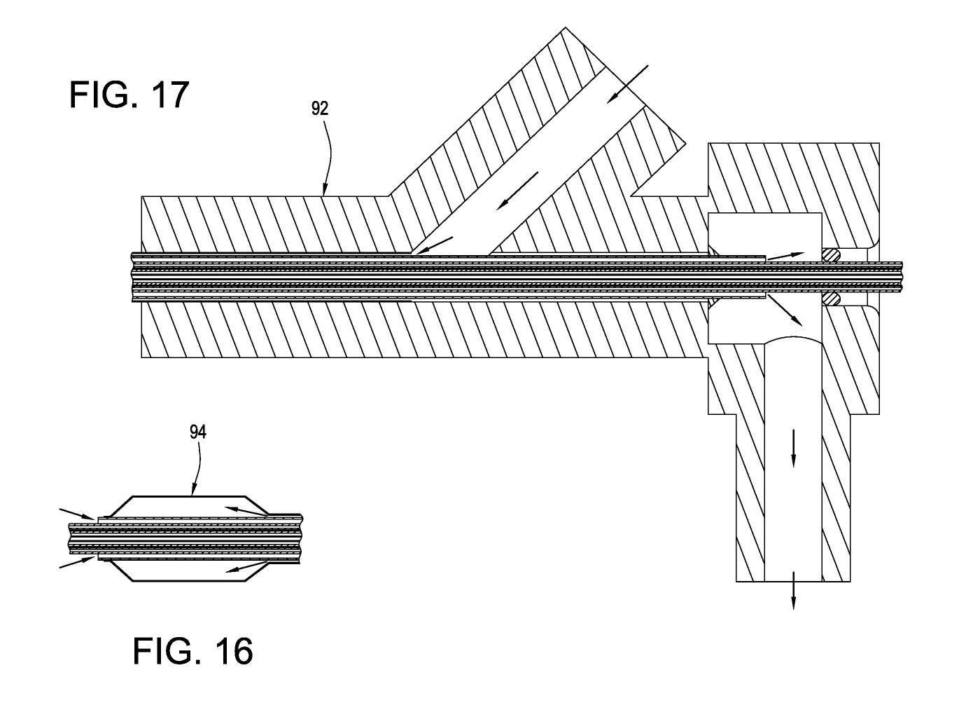

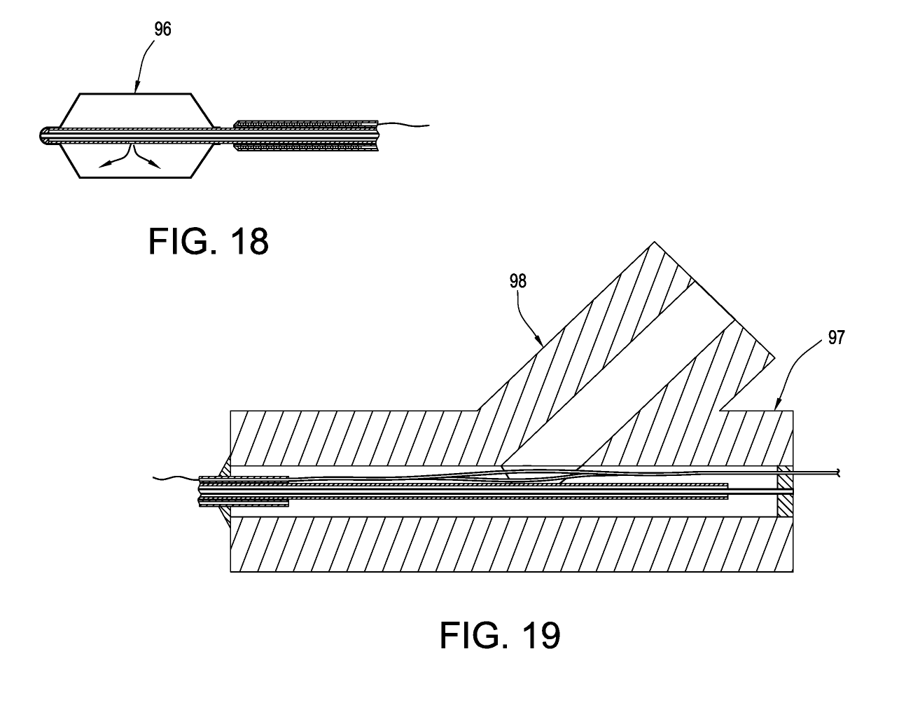

Referring now to FIGS. 15-19 a third embodiment of the apparatus for use in treating a blood vessel is illustrated, designated generally as 90. In this embodiment, the proximal manifold 92 is the same as in the first embodiment for inflating the proximal balloon 94, and the proximal manifold 92 includes the provision for evacuating the blood vessel between the proximal balloon 94 and the distal balloon 96. However, the heating subsystem and distal balloon subsystem are combined as a single piece. In other words, the distal manifold and heating manifold are combined into a single heating/distal manifold 95 containing distal balloon injection port 98; and, heating and sensing conduit 97. A guide wire port may be separate or be integrated with the heating and sensing conduit 97. Evacuation of blood is accomplished by the proximal manifold 92. Thus, although this is a simpler design than the earlier embodiments, evacuation may not be as effective.

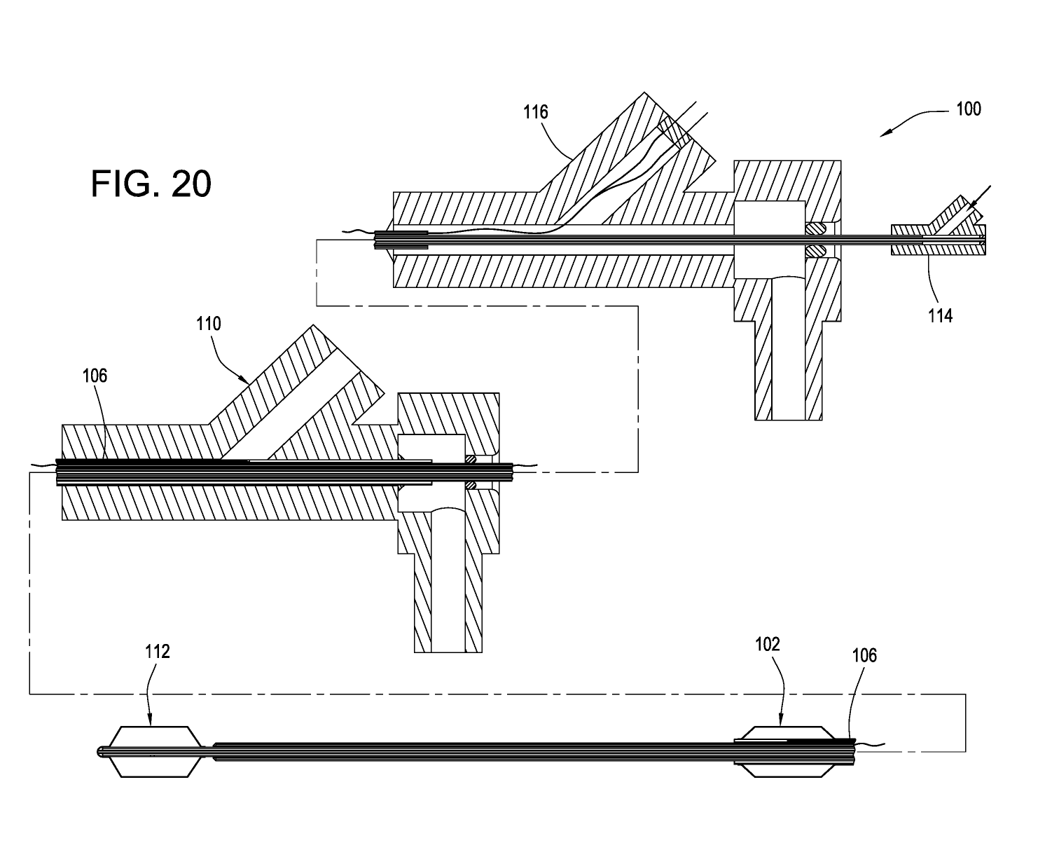

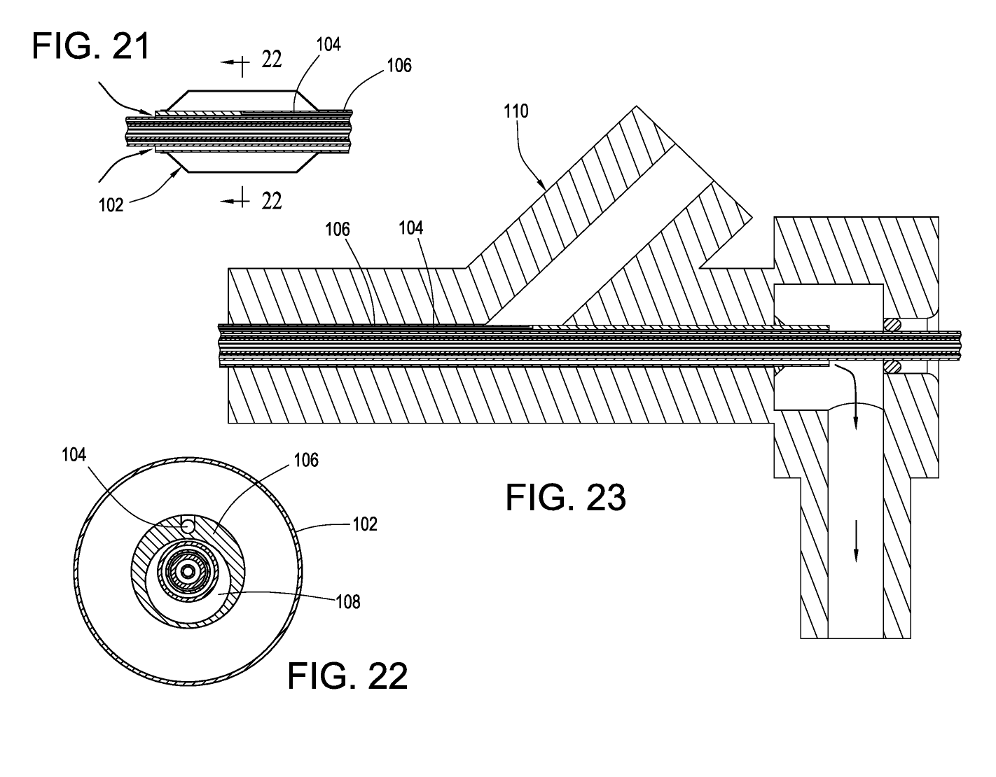

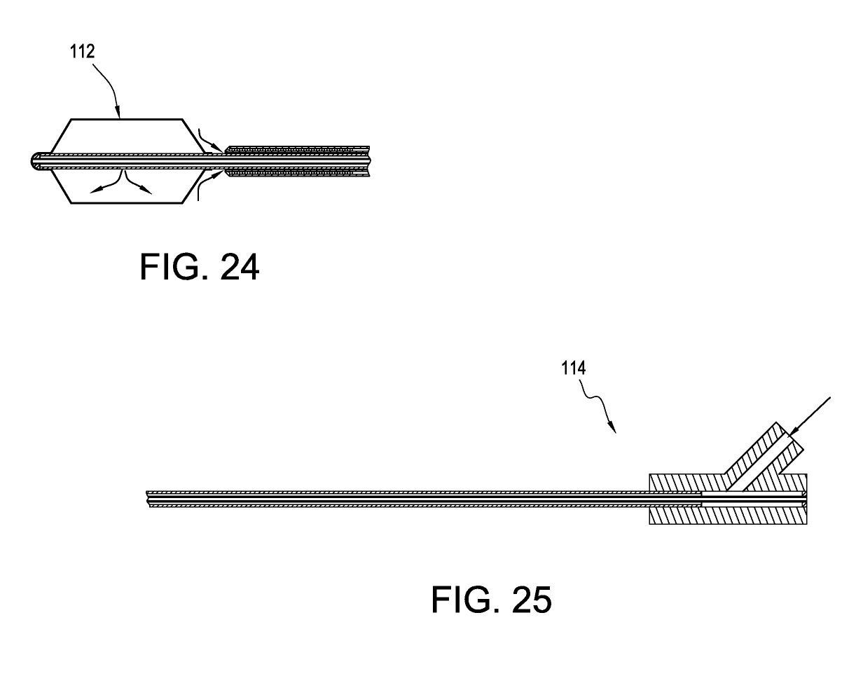

Referring now to FIGS. 20-25, a fourth embodiment of the apparatus for use in treating a blood vessel is illustrated, designated generally as 100. In this embodiment, as shown in FIGS. 21-23, the proximal balloon 102 is inflated by the introduction of fluid from via a lumen 104 formed in tube 106. A larger lumen 108 provides access to the other various components in the ablating apparatus as discussed above. It also provides an evacuation path for the proximal side. The proximal manifold 110, including the various injection tube systems, are shown in FIG. 23. An enlarged section showing the distal balloon 112 is shown in FIG. 24. FIG. 25 illustrates the distal manifold 114. Evacuation takes place on both the proximal and distal sides, via the proximal manifold 110 and the ablating manifold 116, respectively.

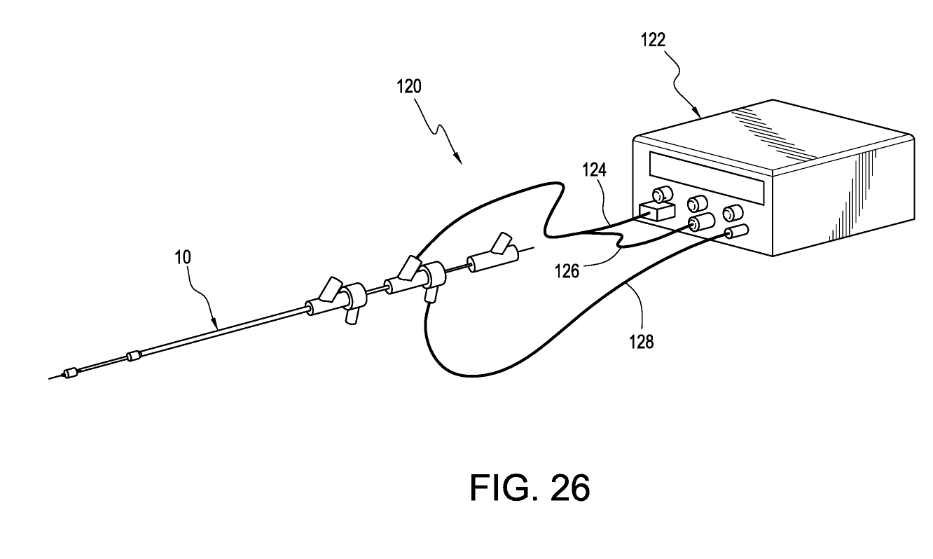

Referring now to FIG. 26 an ablating system 120 is illustrated using the ablating apparatus discussed above. This overall blood vessel treatment system may include a multi-functional control system 122 that provides a source of power for the ablation element (power connection 126), temperature sensing (temperature monitoring connection 124), and pressure sensing (pressure sensing connection 128). FIG. 26 shows one pressure sensing connection 128. However, it is understood that in other embodiments there can be connections to multiple evacuation ports.

Thus, vacuum pressure for blood and other fluids can be monitored to insure proper shrinkage of the vessel. The ablating system is typically configured so that it will not energize the ablation element unless the proper vacuum level has been achieved and will discontinue energy flow if the vacuum level drops below a predetermined threshold.

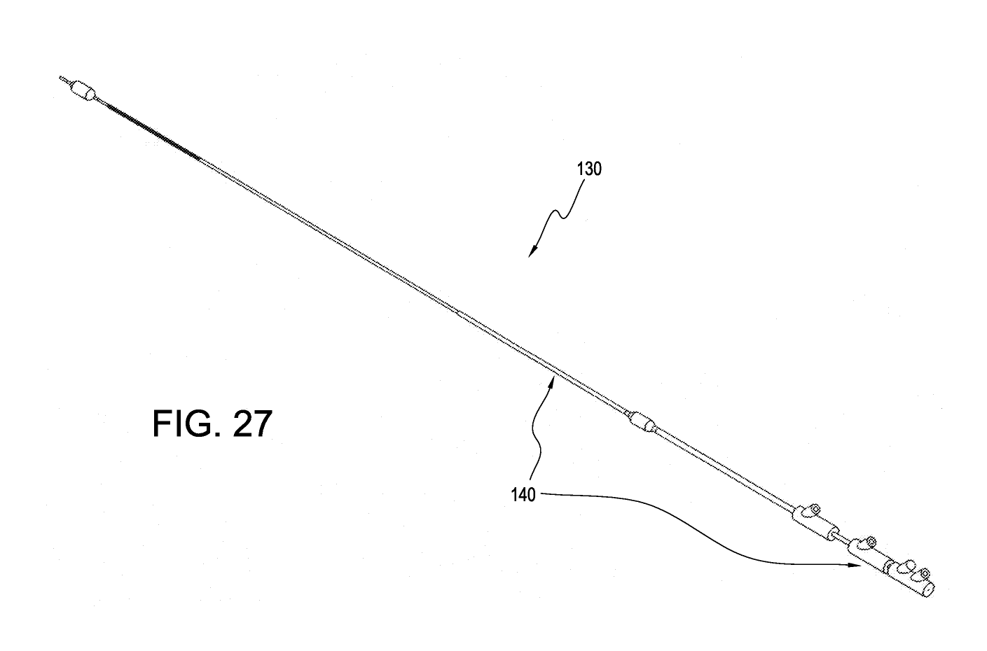

Referring now to FIGS. 27-28 a fifth embodiment is illustrated, designated generally as 130, in which a moveable fluid evacuation subsystem 140 is utilized to extract fluid 33 along the entire length of the treatment area instead of at fixed locations. The fluid evacuation subsystem 140 includes an evacuation manifold 141. An evacuation tubing 142 is attached to the evacuation manifold 141. The proximal manifold 143 and distal manifold 144 are configured to receive the balloon inflating fluid 32 to inflate the proximal balloon 145 and distal balloon 146, respectively.

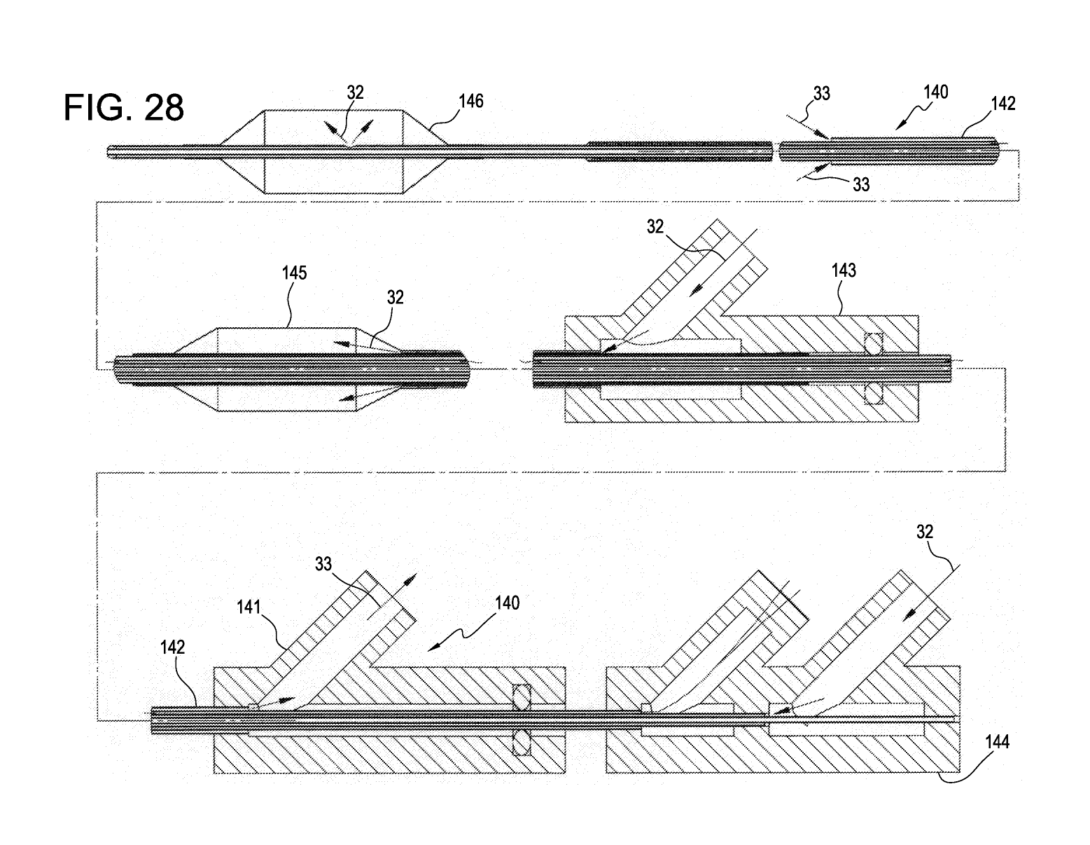

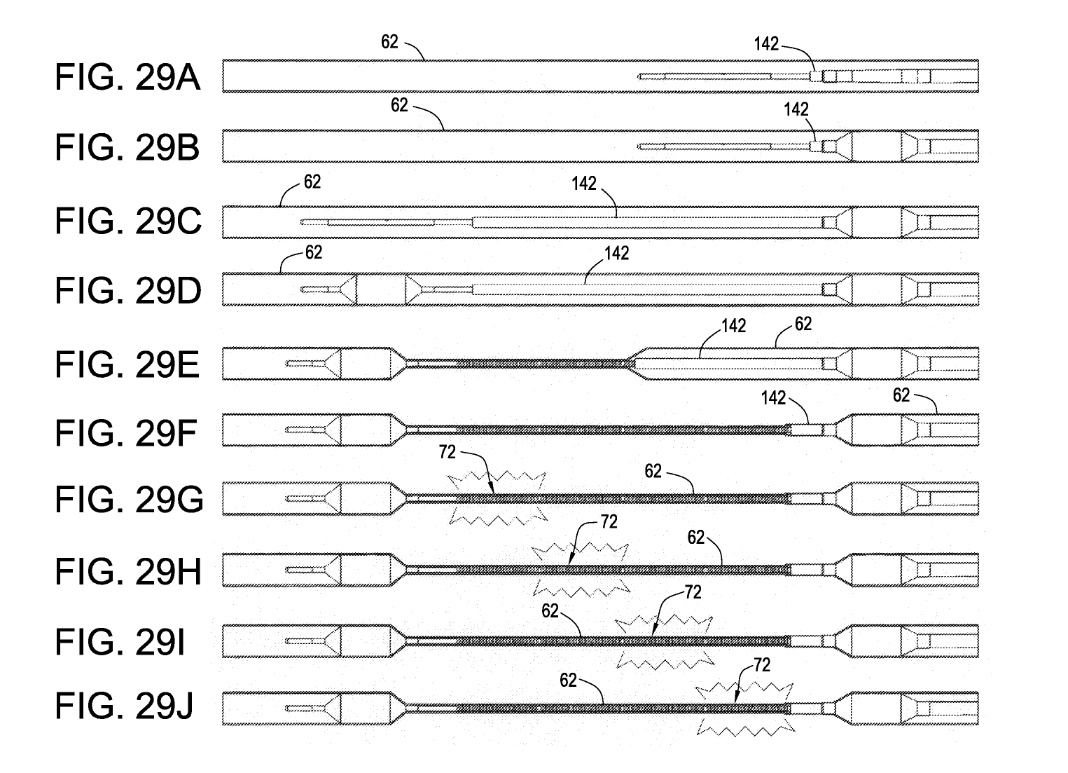

FIGS. 29A-29J illustrate the method of operating the fifth embodiment having a moveable fluid evacuation subsystem 140 capable of extracting fluid more effectively and efficiently.

Referring to FIGS. 29A-D the method for treating a blood vessel includes an initial step of positioning a proximal balloon and a distal balloon of an ablating apparatus, to selected spaced locations within a blood vessel to be ablated. These setup steps include:

a) inserting the ablating apparatus to an initial position within the blood vessel (FIG. 29A);

b) inflating the proximal balloon to abut blood vessel walls of the blood vessel (FIG. 29B);

c) positioning the distal balloon to a distal location of the selected spaced locations (FIG. 29C); and,

d) inflating the distal balloon to abut blood vessel walls of the blood vessel (FIG. 29D).

As shown in FIG. 29E-F, the fluid is evacuated through the evacuation tubing 142. The evacuation tubing 142 is slid along the apparatus between the distal balloon and the proximal balloon to evacuate the fluid between the proximal balloon and distal balloon. Sliding the evacuation tubing 142 along the apparatus prevents the tip of the tubing from becoming clogged by the collapsed blood vessel wall, resulting in more effective fluid evacuation.

The ablating element is then powered. As shown in FIGS. 29G-29J the ablating element segments are powered sequentially to ablate the blood vessel.

Other embodiments and configurations may be devised without departing from the spirit of the invention and the scope of the appended claims. For example, although the present invention has been described as being utilized with balloons it is understood that alternate proximal and distal devices can be utilized to position the ablating element and evacuate the blood vessel therebetween.

1. An ablating apparatus for use in treating a blood vessel, comprising:
a) a proximal balloon subsystem including a proximal balloon positionable to a selected first location within a blood vessel to be ablated;
b) a distal balloon subsystem including a distal balloon positionable to a selected second location within the blood vessel to be ablated;
c) an ablating subsystem including an ablating element configured to be positioned between the proximal balloon and the distal balloon within the blood vessel to be ablated; and,
d) an evacuation port configured to evacuate fluid between the proximal balloon and the distal balloon.

2. The ablating apparatus of claim 1 wherein:
a) said proximal balloon subsystem comprises:
i) a proximal manifold configured to receive balloon inflating fluid;
ii) a proximal balloon injection tube system attached to the proximal manifold for transmitting balloon inflating fluid; and,
iii) said proximal balloon positionable to a selected first location within a blood vessel to be ablated, in fluid communication with said proximal balloon injection tube system;
b) said distal balloon subsystem comprises:
i) a distal manifold configured to receive balloon inflating fluid;
ii) a distal balloon injection tube system attached to the distal manifold for transmitting balloon inflating fluid; and,
iii) said distal balloon positionable to a selected second location within the blood vessel to be ablated, in fluid communication with said distal balloon injection tube system;
c) said ablating subsystem comprises:
i) an ablating manifold;
ii) an ablating element tube system connected to said ablating manifold;
iii) said ablating element operatively connected to a distal end of said ablating element tube system to ablate the blood vessel to be treated; and,
iv) a temperature monitoring element operatively connected to a distal end of said ablating element tube system,
wherein said distal balloon injection tube system is positioned within said ablating element tube system and within said ablating manifold;
wherein said ablating element tube system is positioned within the proximal balloon injection tube system,
wherein either said proximal manifold, the ablating manifold or both the proximal manifold and ablating manifold include an evacuation port configured to evacuate fluid within the blood vessel between the proximal balloon and distal balloon.

3. The ablating apparatus of claim 1, wherein said evacuation port is configured to provide sufficient suction to enable the blood vessel to shrink.

4. The ablating apparatus of claim 1, further comprising a guide wire.

5. The ablating apparatus of claim 1, wherein the ablating element is a heating element, configured to be positioned between the proximal balloon and the distal balloon within the blood vessel to be ablated.

6. A method for treating a blood vessel, comprising:
a) positioning a proximal balloon and a distal balloon of an ablating apparatus, to selected spaced locations within a blood vessel to be ablated, the ablating apparatus including an ablating element positioned between the proximal balloon and the distal balloon within the blood vessel;
b) inflating said proximal balloon and said distal balloon to abut blood vessel walls of the blood vessel at said selected spaced locations;
c) evacuating fluid within the blood vessel between the proximal balloon and distal balloon; and,
d) powering said ablating element to ablate the blood vessel to be treated, between said proximal balloon and said distal balloon.

7. The method of claim 6, wherein the step of positioning a proximal balloon and a distal balloon of an ablating apparatus, to selected spaced locations within a blood vessel to be ablated includes the setup steps of:
a) inserting the ablating device to an initial position within the blood vessel;
b) inflating the proximal balloon to abut blood vessel walls of the blood vessel;
c) positioning said distal balloon to a distal location of said selected spaced locations; and,
d) inflating the distal balloon to abut blood vessel walls of the blood vessel.

8. The method of claim 6, wherein the step of powering said ablating element to ablate the blood vessel to be treated, comprises:
sliding said ablating element along said ablating apparatus between said distal balloon and said proximal balloon to ablate the blood vessel.

9. The method of claim 6, wherein the step of powering said ablating element to ablate the blood vessel to be treated, comprises:
powering segmented portions of said ablating element between said distal balloon and said proximal balloon to ablate the blood vessel for selected periods of time.

10. The method of claim 6, wherein the step of powering said ablating element to ablate the blood vessel to be treated, comprises:
powering entire ablating element between said distal balloon and said proximal balloon to ablate the blood vessel for selected periods of time.

11. The method of claim 6, wherein the step of evacuating the fluid from the treatment area, comprises:
sliding the nozzle of the evacuation subsystem along the length of the treatment area while performing the fluid evacuation.

12. An ablating system for use in treating a blood vessel, comprising:
a) an ablating apparatus, comprising:
i) a proximal balloon subsystem including a proximal balloon positionable to a selected first location within a blood vessel to be ablated;
ii) a distal balloon subsystem including a distal balloon positionable to a selected second location within the blood vessel to be ablated;
iii) an ablating subsystem including an ablating element configured to be positioned between the proximal balloon and the distal balloon within the blood vessel to be ablated; and,
iv) an evacuation port configured to evacuate fluid between the proximal balloon and the distal balloon; and,
b) a multi-functional control system configured to provide power for said ablating element; receive the temperature monitoring indications from the ablating element; and, receive pressure sensing indications from the evacuation port.
